(19) United States
(12) Patent Application Publication
(10) Pub. No.: US 2012/0053417 A1
(43) Pub. Date: Mar. 1, 2012

(54) ENDOSCOPE AND HARDNESS ADJUSTER

(76) Inventors: Shinichi YAMAKAWA, Ashigarakami-gun (JP); Kimitake Fukushima, Ashigarakami-gun (JP); Takayuki Nakamura, Ashigarakami-gun (JP); Maki Saito, Ashigarakami-gun (JP); Shozo Iyama, Ashigarakami-gun (JP); Atsuhiko Ishihara, Ashigarakami-gun (JP); Takayuki Iida, Ashigarakami-gun (JP)

(21) Appl. No.: 13/221,518

(22) Filed: Aug. 30, 2011

(30) Foreign Application Priority Data
Aug. 31, 2010 (JP) 2010-194379

(51) Int. Cl. A61B 1/00

(52) U.S. Cl. 600/144

(57) ABSTRACT

The endoscope includes: a hardness adjusting device which changes the flexibility of the flexible portion of an endoscope insertion unit; and a handling member that handles the hardness adjusting device. The handling member is positioned in a region in which the handling member can be operated with one hand of an operator holding the handheld operation unit of the endoscope, the region being located in an upper portion of the handheld operation unit. The contact spring is extended from the front end of the handheld operation unit to the upper portion of the handheld operation unit, the front end of the handheld operation unit continuing to the flexible portion.

### BACKGROUND OF THE INVENTION

1. Field of the Invention

The present invention generally relates to an endoscope and a hardness adjuster, and more particularly, to an endoscope and a hardness adjuster that enable adjustment of the flexibility of the flexible portion in an endoscope insertion unit.

2. Description of the Related Art

Conventionally, medical examinations using endoscopes have been widely conducted in the field of medicine. Particularly, an imaging element such as a CCD is built in the top end of the insertion unit of an endoscope to be inserted into a body cavity, and captures images inside the body cavity. Signal processing is then performed on the images by a processor device, and is displayed on a monitor. A physician observes the images, and uses the images for diagnoses. Alternatively, a treatment tool is inserted through a channel for treatment tool insertion. With such a treatment tool, samples are collected, or polypectomy is performed, for example.

An endoscope normally includes a main operation unit that is held and operated by a practitioner (hereinafter referred to simply as the operator), and an insertion unit that is connected to the main operation unit and is to be inserted into a body cavity or the like. The endoscope is roughly formed by extending a universal cord from the main operation unit to a connector unit or the like. The universal cord is extended from the main operation unit, and the other end of the universal cord is detachably connected to a light source device (a light source device and a processor).

The insertion unit of the endoscope has a flexible portion having flexibility so that the insertion unit can be inserted into an insertion path having complicated bends. However, because of the flexibility, the top end of the insertion unit is not stabilized in one direction, and therefore, it is difficult to insert the insertion unit in an intended direction. In some cases, the shape of the insertion unit is preferably kept as it is in a body cavity, so as to perform some treatment or observation.

On the other hand, Japanese Patent No. 3,717,559 discloses an endoscope in which the hardness of the flexible portion of the insertion unit can be adjusted by changing the compressed state of a coil pipe placed inside the flexible portion of the endoscope insertion unit. The compressed state of the coil pipe is changed by pulling a pulled wire that is inserted through the coil pipe and has the top end thereof connected to the coil pipe. The pulled wire is pulled by turning a flexibility adjusting knob attached to the rear-end side of the operation unit. The hardness of the coil pipe varies when the coil pipe is compressed.

Japanese Patent Publication Laid-Open No. 2004-209267 discloses an endoscope in which the flexible portion of the insertion unit can be adjusted from a high-flexibility state to a low-flexibility and hardened state by changing the compressed state of a hardness changing coil in accordance with the distance over which a hardness changing wire is moved. The hardness changing wire is moved by turning a hardness adjusting knob attached to the front end of the operation unit. The hardness changing coil is connected to the hardness changing wire, and is placed inside the flexible portion.

Normally, an endoscope operator (the operator) holds the operation unit with his/her left hand and the flexible portion with his/her right hand, when carrying out procedures such as insertion, diagnosis, and treatment. A hardness variable pulling unit and a hardness adjusting device are provided at the base of the endoscope operation unit in any of the above described conventional endoscopes. Therefore, to adjust the hardness of the flexible portion, the operator has to hold and turn a knob that has almost the same diameter as the external diameter of the base of the flexible portion in the palm of his/her hand.

Since the operator holds the operation unit with his/her left hand and the flexible portion with his/her right hand, the operator has to temporarily take his/her right hand off the flexible portion, and turn the knob with the right hand. By endoscopic techniques, particularly in a case where the large or small intestine is to be observed, the position of an inserted endoscope is kept by the balance between the operation force of the right hand acting on the flexible portion and the reaction force from the intestines. Therefore, if the operator temporarily takes his/her right hand off the flexible portion, the position of the inserted endoscope changes, which results in a problem.

If the hardness can be adjusted only with the left hand without release of the flexible portion from the right hand, the operability of the endoscope can be greatly increased. Also, the strength of the pulling mechanism needs to be secured while the hardness adjusting device is being operated only with the left hand.

### SUMMARY OF THE INVENTION

The present invention has been made in view of the above circumstances, and the object thereof is to provide an endoscope and a hardness adjuster that enable one-hand adjustment of the hardness of the flexible portion of the endoscope, and secures the strength of the pulling mechanism during the adjustment.

To achieve the above object, a first aspect of the present invention provides an endoscope which includes: a hardness adjusting device which changes the flexibility of the flexible portion of an endoscope insertion unit; and a handling member that handles the hardness adjusting device. The handling member is positioned in the region in which the handling member can be operated with one hand of an operator holding the handheld operation unit of the endoscope, the region being located in an upper portion of the handheld operation unit.

With this structure, the operator can operate the hardness adjusting device while holding the handheld operation unit only with his/her left hand.

According to a second aspect of the present invention, the hardness adjusting device includes: a hardness adjusting member which is placed in the flexible portion of the endoscope insertion unit, and is capable of changing the flexibility of the flexible portion; a hardness changing device which acts on the hardness adjusting member and changes the hardness of the hardness adjusting member; and a drive device which drives the hardness changing device. The handling member is a handle lever which handles the drive device.

According to a third aspect of the present invention, the hardness adjusting member is a contact spring, the hardness changing device is a wire which is inserted through the contact spring, and the drive device is a wire pulling device which pulls the wire.

According to a fourth aspect of the present invention, the top end of the wire is fixed to the top end of the contact spring on the top end side of the flexible portion, and the rear end of the contact spring is fixed to a contact spring fixing device.

According to a fifth aspect of the present invention, the wire pulling device is placed in the upper portion of the handheld operation unit of the endoscope, the contact spring fixing device is placed near the wire pulling device, and the contact spring is extended from the front end of the handheld operation unit to the upper portion of the handheld operation unit, the front end of the handheld operation unit continuing to the flexible portion.

As described above, the wire pulling device is placed in the upper portion of the handheld operation unit, and the contact spring is extended to the upper portion of the handheld operation unit. Accordingly, the region that needs to have mechanical strength can be made smaller.

According to a sixth aspect of the present invention, the diameter of the portion of the contact spring extended to the upper portion of the handheld operation unit is smaller than the diameter of the portion of the contact spring located inside the flexible portion.

As described above, the diameter of the contact spring located inside the handheld operation unit is made smaller. Accordingly, the space inside the handheld operation unit can be made larger, and be effectively used.

To achieve the above object, a seventh aspect of the present invention provides a hardness adjuster which includes: a contact spring which is provided at least inside the flexible portion of an endoscope insertion unit, and is fixed to a contact spring fixing device on a side of the handheld operation unit of the endoscope; a wire which is inserted through the contact spring, and has the top end fixed to the top end of the contact spring; a wire pulling device which pulls the wire; and a handling member which handles the wire pulling device, and is placed in the region in which the handling member can be operated with one hand of an operator holding the handheld operation unit of the endoscope, the region being located in an upper portion of the handheld operation unit.

With this structure, the operator can operate the hardness adjusting device while holding the handheld operation unit only with his/her left hand.

According to an eighth aspect of the present invention, the wire pulling device is placed in the upper portion of the handheld operation unit of the endoscope, the contact spring fixing device is placed near the wire pulling device, and the contact spring is extended from the front end of the handheld operation unit to the upper portion of the handheld operation unit by a predetermined member, the front end of the handheld operation unit continuing to the flexible portion.

As described above, the wire pulling device is placed in the upper portion of the handheld operation unit, and the contact spring is extended to the upper portion of the handheld operation unit. Accordingly, the region that needs to have mechanical strength can be made smaller.

According to a ninth aspect of the present invention, the predetermined member which extends the contact spring from the front end of the handheld operation unit continuing to the flexible portion to the upper portion of the handheld operation unit is a contact spring which has a smaller diameter than the diameter of the contact spring located inside the flexible portion.

According to a tenth aspect of the present invention, the predetermined member which extends the contact spring from the front end of the handheld operation unit continuing to the flexible portion to the upper portion of the handheld operation unit is a metal pipe which has a smaller diameter than the diameter of the contact spring located inside the flexible portion.

As described above, the diameter of the member which extends the contact spring into the handheld operation unit is made smaller. Accordingly, the space inside the handheld operation unit can be made larger, and be effectively used.

According to an eleventh aspect of the present invention, the wire pulling device is a wire pulley which is driven through a gear driven by a handle lever serving as the handling member.

According to a twelfth aspect of the present invention, the wire pulling device is a wire pulley which is driven by an electric motor.

As described above, the wire pulling device may be either manually or electrically driven.

As described above, according to the present invention, the handling member of the hardness adjusting device is placed in the region in which the handling member can be operated with one hand of an operator holding the handheld operation unit of the endoscope, with the region being located in an upper portion of the handheld operation unit. Accordingly, the operator can operate the hardness adjusting device while holding the handheld operation unit only with his/her left hand. Also, the contact spring of the hardness adjusting device is extended to the upper portion of the handheld operation unit. Accordingly, the region that needs to have mechanical strength can be made smaller. Further, the diameter of the extended portion is reduced, so that the space inside the handheld operation unit can be made larger and be effectively used.

### BRIEF DESCRIPTION OF THE DRAWINGS

FIG. 1 is a schematic view showing the structure of an embodiment of an endoscope including a hardness adjuster according to the present invention;

FIG. 2 is a longitudinal cross-sectional view of the internal structure of the endoscope;

FIG. 3 is a cross-sectional view of the handheld operation unit, showing the structure of the wire pulling unit;

FIG. 4 is an enlarged cross-sectional view of the handheld operation unit;

FIG. 5 is another enlarged cross-sectional view of the handheld operation unit;

FIG. 6 is yet another enlarged cross-sectional view of the handheld operation unit;

FIG. 7 is a cross-sectional view illustrating a first method of electrically rolling up a wire; and

FIG. 8 is a cross-sectional view illustrating a second method of electrically rolling up a wire.

### DETAILED DESCRIPTION OF THE PREFERRED EMBODIMENTS

The following is a detailed description of an endoscope and a hardness adjuster according to the present invention, with reference to the accompanying drawings.

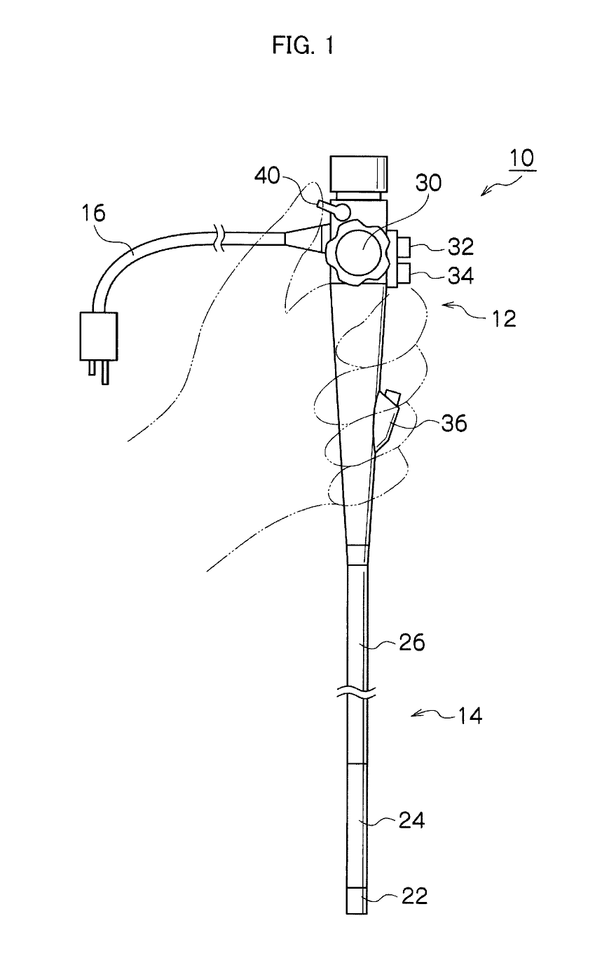

FIG. 1 is a schematic view showing the structure of an embodiment of an endoscope that includes a hardness adjuster according to the present invention.

As shown in FIG. 1, the endoscope 10 of this embodiment includes a handheld operation unit 12 and an insertion unit 14 joined to the handheld operation unit 12. An operator holds and handles the handheld operation unit 12 with his/her left hand, and holds the insertion unit 14 with his/her right hand. The operator then puts the insertion unit 14 into a body cavity of a subject, and conducts an observation.

A universal cable 16 is connected to the handheld operation unit 12. Although not shown in the drawing, a LG connector is attached to the top end of the universal cable 16, and the LG connector is detachably connected to a light source device. With this arrangement, illumination light is supplied to an illumination optical system provided at the top end of the insertion unit 14. Also, though not shown in the drawing, an electric connector is connected to the LG connector via the universal cable 16, and the electric connector is detachably connected to an endoscope processor. With this arrangement, the data about an observed image obtained by the endoscope 10 is output to the endoscope processor, and the image is displayed on a monitor device connected to the endoscope processor. Using the displayed image, the operator conducts an observation.

The insertion unit 14 is connected to the top end of the handheld operation unit 12 as shown in FIG. 1. The insertion unit 14 includes a flexible portion 26, a curving portion (an angled portion) 24, and a top end portion 22, when seen from the base end (on the side of the handheld operation unit 12) toward the top end (on the side to be inserted into a body cavity). The curving portion 24 is remotely curved when an angle knob 30 provided on the handheld operation unit 12 is rotated. Accordingly, the top end surface of the top end portion 22 can be made to face in a desired direction.

The handheld operation unit 12 includes: an air/water supply button 32 for supplying air and water from an air/water supply outlet at the top end portion 22 to an area to be examined or the like via an air/water supply channel; a suction button 34 for applying suction from a forceps slit at the top end portion 22 via a forceps channel; and a forceps insertion slot 36 that is an opening continuing to the forceps channel and is designed for the operator to insert forceps therethrough.

The endoscope 10 also includes a hardness adjuster that adjusts the hardness (or changes the flexibility) of the flexible portion 26. As will be described later in detail, a contact spring (a coil) is provided in the flexible portion 26, and a wire is firmly fixed to the contact spring on the top end side of the flexible portion and is inserted through the contact spring fixed to a fixing member on the side of the handheld operation unit 12. The wire is pulled to compress the contact spring and increase the hardness of the contact spring. Accordingly, the hardness of the flexible portion 26 is increased.

A handle lever 40 of a hardness adjusting device to adjust the hardness of the flexible portion 26 is provided on the upper portion of the handheld operation unit 12. When the handle lever 40 is operated, the wire is pulled via a wire pulling unit. Particularly, the handle lever 40 is positioned within such a region that the thumb of the left hand holding the operation unit 12 can reach the handle lever 40, as indicated by the two-dot chain line in FIG. 1.

Further, as will be described later in detail, the wire pulling unit of the hardness adjusting device and the fixing member for the contact spring subjected to the wire pulling force are also provided in the upper portion of the handheld operation unit 12 in this embodiment.

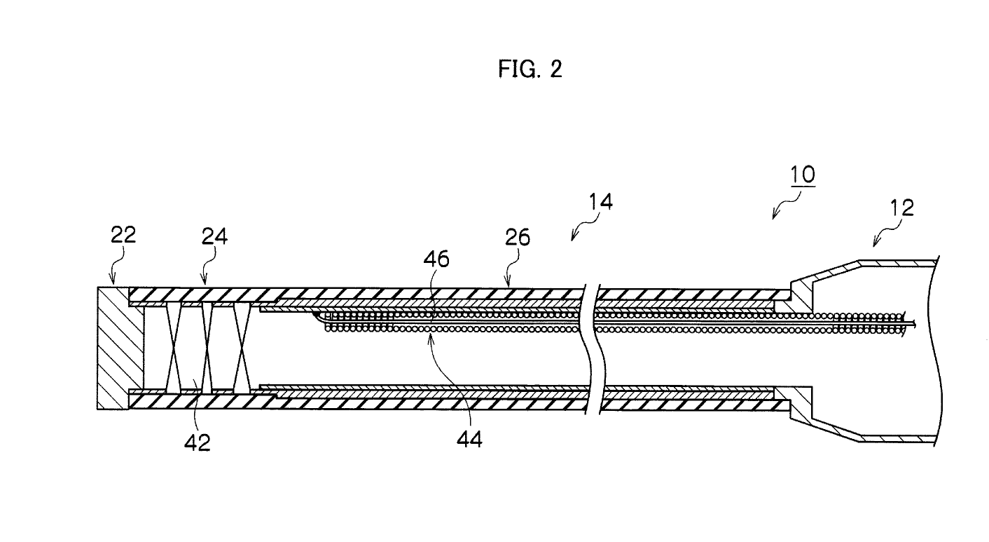

FIG. 2 is a longitudinal cross-sectional view of the structure of the endoscope 10.

As shown in FIG. 2, the curving portion 24 of the insertion unit 14 is formed by a large number of curving pieces 42 (angle members) formed in a ring-like shape. Each two curving pieces 42 are rotatably joined to each other. When the angle knob 30 (see FIG. 1) of the handheld operation unit 12 is turned, the curving portion 24 is curved horizontally or vertically. Accordingly, the top end surface 23 of the top end portion 22 can be made to face in a desired direction.

Also, as shown in FIG. 2, a contact spring (the hardness adjusting coil) 44 forming the hardness adjusting device, and a wire (the hardness adjusting wire) 46 inserted through the contact spring 44 are provided inside the flexible portion 26.

The wire 46 inserted through the contact spring 44 has one end fixed to the top end of the contact spring 44, and has the other end connected to the wire pulling unit that is placed inside the handheld operation unit 12 but is not shown in FIG. 2. As described above, when the handle lever 40 provided on the upper portion of the handheld operation unit 12 is operated, the wire 46 is pulled by the wire pulling unit. As a result, the contact spring 44 is compressed, and is put into a low-flexibility and hardened state. In this manner, the hardness of the flexible portion 26 is increased.

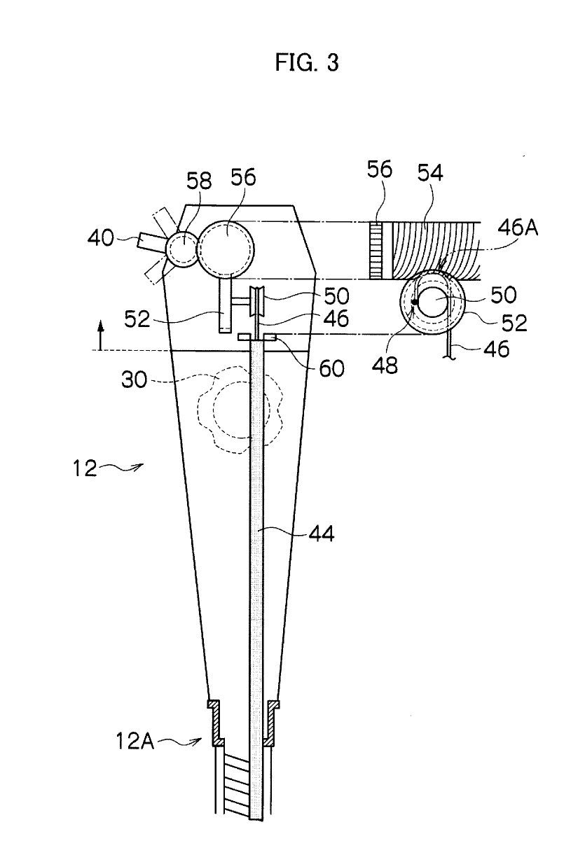

FIG. 3 illustrates the structure of the wire pulling unit. The left half of FIG. 3 is a cross-sectional view of the handheld operation unit 12, and the right half of FIG. 3 is a side view of the wire pulling mechanism seen from the right side of the handheld operation unit 12 in the drawing.

As shown in the left half of FIG. 3, a wire pulley 50 of the wire pulling unit for pulling the wire 46 inserted through the contact spring 44 is provided in the upper portion of the handheld operation unit 12.

The wire 46 is wound around the wire pulley 50, and has its end point 48 fixed to the wire pulley 50. Also, the wire pulley 50 is coaxially connected to a worm wheel (a pulley drive gear) 52.

As shown in the right half of FIG. 3, which is a side view of the wire pulley 50 and its surrounding area seen from the right-hand side, the worm wheel 52 meshes with a worm gear (a reduction gear) 54. A spur gear 56 is coaxially connected to the worm gear 54, and the spur gear 56 meshes with a gear 58 connected to the handle lever 40.

As shown in the left half of FIG. 3, the fixing member 60 that fixes the contact spring 44 is provided in the immediate vicinity of the wire pulley 50 (the wire pulling device) provided in the upper portion of the handheld operation unit 12.

When an operator operates the handle lever 40, the gear 58 connected to the handle lever 40 is driven, and the spur gear 56 is driven accordingly. As a result, the worm gear 54 coaxially connected to the spur gear 56 is driven. The worm wheel 52 is then driven by the worm gear 54, and the wire pulley 50 is rotated to pull the wire 46.

The top end of the wire 46 is fixed to the top end of the contact spring 44, and one end of the contact spring 44 is fixed to the fixing member 60. Therefore, when the wire 46 is pulled, the contact spring 44 is pulled toward the wire pulley 50 of the wire pulling unit, and is compressed with the fixing member 60. Accordingly, the hardness of the contact spring 44 increases.

As described above, in this embodiment, the fixing member 60 that fixes the contact spring 44 is provided in the upper portion of the handheld operation unit 12, to extend the contact spring 44 to the upper portion of the handheld operation unit 12.

Also, the handle lever 40 is designed to be moved up and down, as indicated by broken lines in FIG. 3. When the handle lever 40 is moved upward, the spur gear 56 is driven by the gear 58, and the worm gear 54 is driven with the spur gear 56, and the worm wheel 52 is driven by the worm gear 54. In this manner, the wire pulley 50 is rotated in the roll-up direction of the wire 46, and the wire 46 is pulled to compress the contact spring 44. The hardness of the contact spring 44 then becomes higher, and the hardness of the flexible portion 26 also becomes higher (or the flexibility of the flexible portion 26 becomes lower). When the handle lever 40 is moved downward, the respective gears are driven in the opposite directions of the above, and the wire pulley 50 is rotated in the roll-down direction of the wire 46. The wire 46 is then relaxed, and the contact spring 44 is expanded. Accordingly, the hardness of the contact spring 44 becomes lower, and the hardness of the flexible portion 26 also becomes lower (or the flexibility of the flexible portion 26 becomes higher).

Here, the operation force is transmitted from the handle lever 40 to the worm gear 54 through the gear 58 of the handle lever 40, and is further transmitted to the wire pulley 50 through the worm wheel 52. However, the wire 46 is fixed to the top end of the contact spring 44, and the contact spring 44 is curved and becomes longer when the insertion unit 14 (the flexible portion 26) is curved. Therefore, even when the handle lever 40 is not operated, the wire 46 is pulled toward the wire pulley 50 in a relative manner, and the hardness of the contact spring 44 varies. To prevent the hardness of the contact spring 44 from varying when the insertion portion 14 is curved while the handle lever 40 is not operated and the hardness is zero, the wire 46 has initial slack (an initial extra length) as indicated by the reference numeral 46A in FIG. 3.

When the operator operates the handle lever 40 to increase the hardness of the flexible portion 26, the worm wheel 52 is secured in the current position by the friction between the gear tooth surfaces of the worm gear 54 and the worm wheel 52 even if the operator takes his/her thumb off the handle lever 40. As the worm wheel 52 is secured by the worm gear 54 in this manner, the wire pulley 50 is secured in a desired position, and the wire 46 can be maintained in a pulled state. As described above, the worm gear 54 has the braking function to hold the wire pulled state. The worm gear 54 has a speed reducing function, and is incorporated into the structure to reduce the wire pulling force that acts on the wire 46 and reaches several tens of kilograms of force (kgf) to a smaller operation force.

As described above, according to this embodiment, the wire 46 is pulled to increase the hardness of the contact spring 44. However, the top end of the wire 46 is tightly bonded to the contact spring 44 on the top end side of the flexible portion 26. Therefore, the pulling force for the wire 46 acts, as the compression force for the contact spring 44, on the fixing member 60 of the contact spring 44 located in the upper portion of the handheld operation unit 12.

That is, the wire pulling mechanism and the fixing member 60 of the contact spring 44 are structurally connected to each other, to keep the equilibrium of force. Since the wire pulling force reaches several tens of kilograms of force (kgf), the connecting structure between the wire pulling mechanism and the contact spring fixing unit needs to be strong.

In conventional cases, the contact spring fixing unit is provided at the base of the handheld operation unit 12 (near the region indicated by the reference numeral 12A in FIG. 3). If only the pulling mechanism is provided in the upper portion of the handheld operation unit 12, the structure between the upper portion and the base of the handheld operation unit 12 needs to be made strong.

In this embodiment, on the other hand, the fixing member 60 that fixes the contact spring 44 is provided in the upper portion of the handheld operation unit 12, and the contact spring 44 is extended to the upper portion of the handheld operation unit 12. Accordingly, the length of the structure that needs to be strong can be made remarkably smaller.

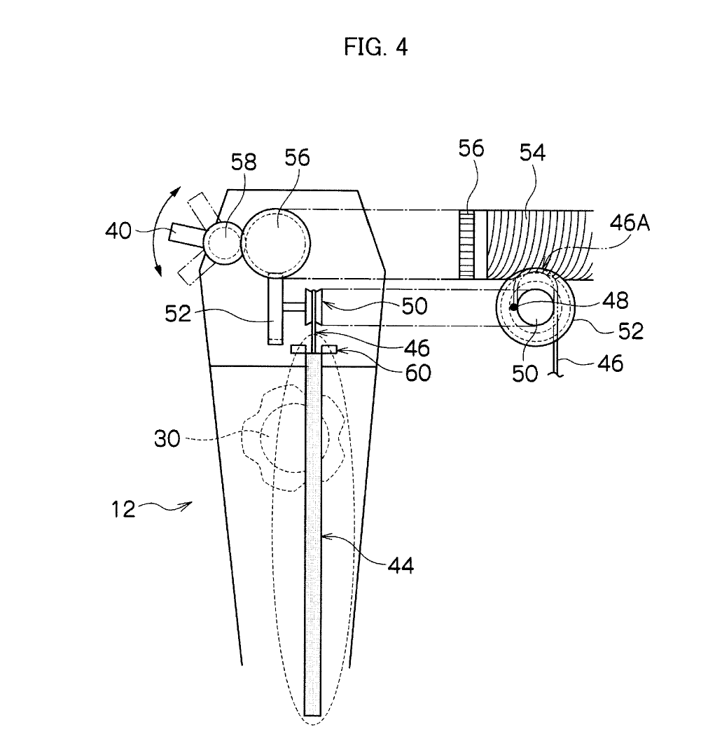

FIG. 4 is an enlarged cross-sectional view of the handheld operation unit 12. As shown in FIG. 4, the contact spring 44 is extended to the location of the fixing member 60 provided in the upper portion of the handheld operation unit 12. In FIG. 4, the contact spring 44 inside the flexible portion 26 is extended to the upper portion of the handheld operation unit 12, without a variation in the diameter of the contact spring 44.

In the handheld operation unit 12, however, the hardness of the contact spring 44 does not need to be varied, and the contact spring 44 only has to transmit the compression force generated from the operation force from the handle lever 40 to the portion of the contact spring 44 located inside the flexible portion 26. Therefore, the portion of the contact spring 44 indicated by a broken line in FIG. 4 inside the handheld operation unit 12 may have a smaller diameter than that of the portion of the contact spring 44 located inside the flexible portion 26.

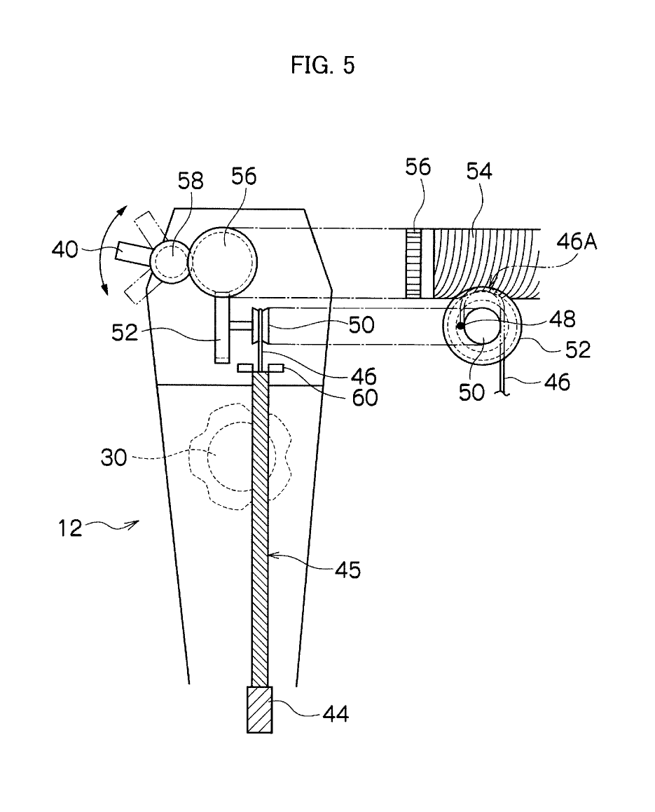

Therefore, inside the handheld operation unit 12, a contact spring 45 having a smaller diameter than that of the contact spring 44 located inside the flexible portion 26 may be used, as shown in FIG. 5. In such a case, the top end of the small-diameter contact spring 45 is connected to the contact spring 44 located inside the flexible portion 26 at the front end of the handheld operation unit 12. The rear end of the small-diameter contact spring 45 is fixed to the fixing member 60. The wire 46 is inserted through the small-diameter contact spring 45. To increase the hardness of the flexible portion 26, the handle lever 40 is operated to pull the wire 46 and push the small-diameter contact spring 45 against the fixing member 60. The compression force generated there is transmitted from the small-diameter contact spring 45 to the contact spring 44 inside the flexible portion 26. As the contact spring 44 inside the flexible portion 26 is compressed, the contact spring 44 becomes harder, and the hardness of the flexible portion 26 increases accordingly.

The diameter of the small-diameter contact spring 45 is larger than the diameter of the wire 46 inserted through the small-diameter contact spring 45, but is smaller than the diameter of the contact spring 44 located inside the flexible portion 26.

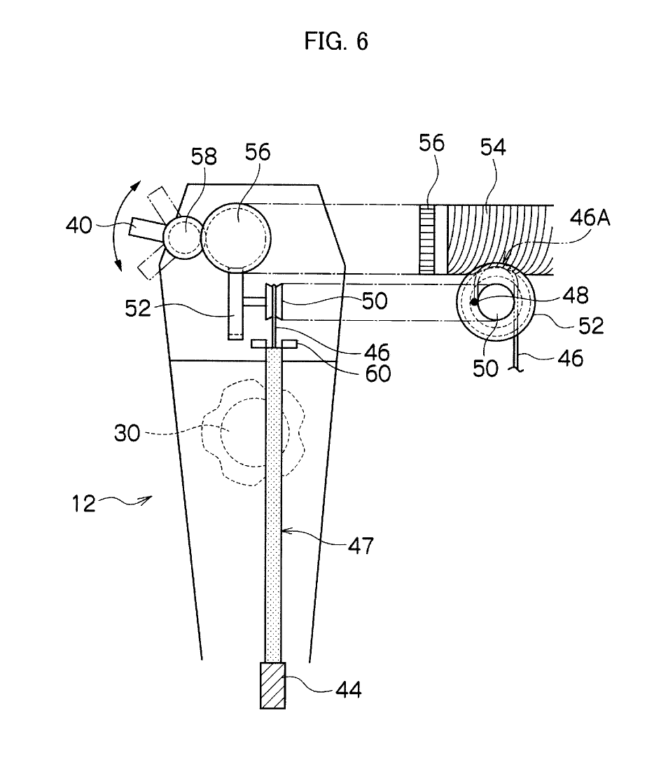

Inside the handheld operation unit 12, the contact spring 45 does not needs to be curved. Therefore, instead of the contact spring 45, a small-diameter metal pipe 47 that is hardly curved may be used as shown in FIG. 6.

The wire 46 is inserted through the small-diameter metal pipe 47. As shown in FIG. 6, the upper end of the metal pipe 47 is fixed to the fixing member 60, and the lower end of the metal pipe 47 is connected to the contact spring 44 located inside the flexible portion 26.

To increase the hardness of the flexible portion 26, the handle lever 40 is operated to pull the wire 46 and push the small-diameter metal pipe 47 against the fixing member 60. The compression force generated there is transmitted from the small-diameter metal pipe 47 to the contact spring 44 located inside the flexible portion 26. The contact spring 44 inside the flexible portion 26 is then compressed, and the contact spring 44 becomes harder. As a result, the hardness of the flexible portion 26 increases.

The space inside the handheld operation unit 12 can be effectively used by employing one of the small-diameter contact spring 45 and the small-diameter metal pipe 47 as described above.

In each of the above described examples, the wire pulling unit is of a manual type. However, the wire pulling unit is not limited to a manual type, and may be of an electrically-driven type as described below.

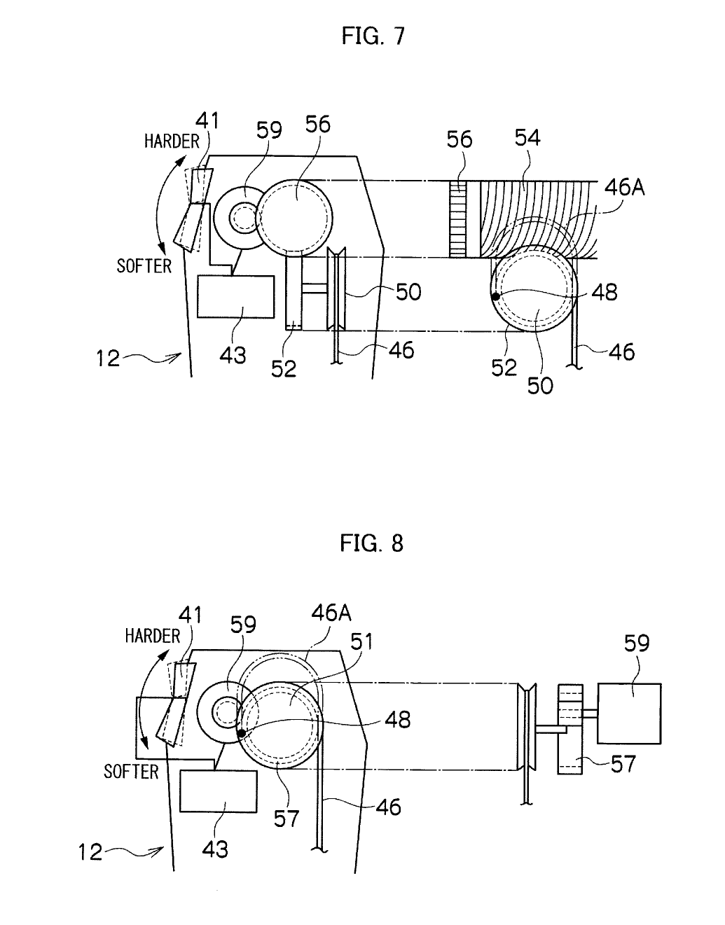

FIG. 7 illustrates a first method of electrically rolling up a wire. The left half of FIG. 7 is an enlarged cross-sectional view of the upper portion of the handheld operation unit 12. The right half of FIG. 7 is a side view of the wire pulling unit seen from the right-hand side.

Like the above described the manual type, the first electrically wire roll-up method illustrated in FIG. 7 involves the wire pulley 50 that rolls up the wire 46, the worm wheel 52 coaxially fixed to the wire pulley 50, the worm gear 54 that drives the worm wheel 52, the spur gear 56 coaxially fixed to the worm gear 54, and the like.

In the case of the manual type, the spur gear 56 is driven by the gear 58 (see FIG. 3, for example) driven by the handle lever 40. In the case of the electrically-driven type, however, the spur gear 56 is driven by a motor 59.

It should be noted that the motor 59 is not particularly limited, and may be a DC motor, a stepping motor, or a servo motor, for example. The motor 59 includes a hardness determining function that determines the hardness of the contact spring 44 of the flexible portion 26. As in the case of the manual type, the worm gear 54 includes the braking function that maintains the hardness of the contact spring 44 at a certain value.

The motor 59 is controlled by a control system 43 through an operation button 41. Like the handle lever 40 of the manual type, the operation button 41 is provided in such a position that the operation button 41 can be operated only with the left hand (or the thumb of the left hand, for example). Such a position is located in the upper portion of the handheld operation unit 12. In the drawing, the operation button 41 is formed by a seesaw-like button. When one end of the operation button 41 is pressed, the hardness increases. When the other end is pressed, the hardness decreases. However, the shape of the operation button 41 is not particularly limited, and the operation button 41 may have any shape, as long as the operation button 41 can be operated with the thumb of one hand.

FIG. 8 illustrates a second method of electrically rolling up a wire. The left half of FIG. 8 is an enlarged cross-sectional view of the upper portion of the handheld operation unit 12. The right half of FIG. 8 is a side view of the wire pulling unit seen from the right-hand side.

By the second electrical wire roll-up method illustrated in FIG. 8, the pulley that rolls up the wire 46 is driven directly by a motor.

As shown in FIG. 8, the wire 46 is rolled up by a wire pulley 51. The end point 48 of the wire 46 is fixed to the wire pulley 51, and is wound around the wire pulley 51, with initial slack 46A being kept. As shown in FIG. 8, the wire pulley 51 is driven directly by a motor in this example, the wire pulley 51 has a larger diameter than the wire pulley 50 of each of the foregoing examples.

A spur gear 57 is coaxially connected to the wire pulley 51, and the spur gear 57 is driven by the motor 59.

As in the foregoing example, the motor 59 is not particularly limited, and may be a DC motor, a stepping motor, a servo motor, or the like. In this example, however, a worm gear is not used, and therefore, the motor 59 preferably includes the braking function as well as the hardness determining function.

The motor 59 is controlled by the operation button 41 through the control system 43. As in the foregoing example, the operation button 41 is provided in such a position that the operation button 41 can be operated only with the left hand (or the thumb of the left hand, for example). Such a position is located in the upper portion of the handheld operation unit 12.

As described above, the wire pulling unit of the hardness adjusting device is provided in an upper portion of the handheld operation unit, and the operation device (the handle lever or the operation button) is provided in such a region that the operation device can be reached by the thumb of one hand holding the handheld operation unit, with the region being located in the upper portion of the handheld operation unit. The contact spring is extended to the upper portion of the handheld operation unit, and is then fixed. Accordingly, the distance from the contact spring fixing unit to the wire pulley in the handheld operation unit can be made much shorter, and the region that needs to have mechanical strength can be made smaller. Also, a hardness adjusting operation can be performed only with one hand (the left hand, for example).

The extended portion of the contact spring has a smaller diameter than the external diameter of the contact spring located inside the flexible portion. Accordingly, the space inside the handheld operation unit can be effectively used. Also, the extended portion of the contact spring is formed by a metal pipe having a smaller diameter than the external diameter of the contact spring located inside the flexible portion. Accordingly, the space inside the handheld operation unit can also be effectively used.

The variable hardness adjusting unit, the wire pulling unit, and the contact spring fixing unit are collectively provided in the upper portion of the handheld operation unit. For example, the portion of the handheld operation unit located above the contact spring fixing member shown in FIG. 3 and the like is regarded as an independent module such as a function expanding module. With this arrangement, maintenance becomes easier than in a case where the variable hardness adjusting unit, the wire pulling unit, and the contact spring fixing unit are provided between the handheld operation unit and the flexible portion.

Also, in a case where an electric motor or the like is used for the wire pulling unit, adverse influence such as electromagnetic noise on a CHA cable can be minimized by shielding the upper portion of the handheld operation unit shown in FIG. 7 or 8 as a module.

Endoscopes and hardness adjusters according to the present invention have been described in detail so far. However, the present invention is not limited to the above examples, and various changes and modifications may of course be made to those examples without departing from the scope of the invention.

What is claimed is:

1. An endoscope comprising:
a hardness adjusting device which changes flexibility of a flexible portion of an endoscope insertion unit; and
a handling member that handles the hardness adjusting device,
the handling member being positioned in a region in which the handling member can be operated with one hand of an operator holding a handheld operation unit of the endoscope, the region being located in an upper portion of the handheld operation unit.

2. The endoscope according to claim 1, wherein
the hardness adjusting device includes: a hardness adjusting member which is placed in the flexible portion of the endoscope insertion unit, and is capable of changing the flexibility of the flexible portion; a hardness changing device which acts on the hardness adjusting member and changes hardness of the hardness adjusting member; and a drive device which drives the hardness changing device, and
the handling member is a handle lever which handles the drive device.

3. The endoscope according to claim 2, wherein
the hardness adjusting member is a contact spring,
the hardness changing device is a wire which is inserted through the contact spring, and
the drive device is a wire pulling device which pulls the wire.

4. The endoscope according to claim 3, wherein
a top end of the wire is fixed to a top end of the contact spring on a top end side of the flexible portion, and
a rear end of the contact spring is fixed to a contact spring fixing device.

5. The endoscope according to claim 4, wherein
the wire pulling device is placed in the upper portion of the handheld operation unit of the endoscope,
the contact spring fixing device is placed near the wire pulling device, and
the contact spring is extended from a front end of the handheld operation unit to the upper portion of the handheld operation unit, the front end of the handheld operation unit continuing to the flexible portion.

6. The endoscope according to claim 5, wherein a diameter of a portion of the contact spring extended to the upper portion of the handheld operation unit is smaller than a diameter of a portion of the contact spring located inside the flexible portion.

7. A hardness adjuster comprising:
a contact spring which is provided at least inside a flexible portion of an endoscope insertion unit, and is fixed to a contact spring fixing device on a side of a handheld operation unit of the endoscope;
a wire which is inserted through the contact spring, and has a top end fixed to a top end of the contact spring;
a wire pulling device which pulls the wire; and
a handling member which handles the wire pulling device, and is placed in a region in which the handling member can be operated with one hand of an operator holding the handheld operation unit of the endoscope, the region being located in an upper portion of the handheld operation unit.

8. The hardness adjuster according to claim 7, wherein
the wire pulling device is placed in the upper portion of the handheld operation unit of the endoscope,
the contact spring fixing device is placed near the wire pulling device, and
the contact spring is extended from a front end of the handheld operation unit to the upper portion of the handheld operation unit by a predetermined member, the front end of the handheld operation unit continuing to the flexible portion.

9. The hardness adjuster according to claim 8, wherein
the predetermined member which extends the contact spring from the front end of the handheld operation unit continuing to the flexible portion to the upper portion of the handheld operation unit is a contact spring which has a smaller diameter than a diameter of the contact spring located inside the flexible portion.

10. The hardness adjuster according to claim 8, wherein the predetermined member which extends the contact spring from the front end of the handheld operation unit continuing to the flexible portion to the upper portion of the handheld operation unit is a metal pipe which has a smaller diameter than a diameter of the contact spring located inside the flexible portion.

11. The hardness adjuster according to claim 7, wherein
the wire pulling device is a wire pulley which is driven through a gear driven by a handling lever serving as the handling member.

12. The hardness adjuster according to claim 8, wherein
the wire pulling device is a wire pulley which is driven through a gear driven by a handling lever serving as the handling member.

13. The hardness adjuster according to claim 9, wherein
the wire pulling device is a wire pulley which is driven through a gear driven by a handling lever serving as the handling member.

14. The hardness adjuster according to claim 10, wherein
the wire pulling device is a wire pulley which is driven through a gear driven by a handling lever serving as the handling member.

15. The hardness adjuster according to claim 7, wherein
the wire pulling device is a wire pulley which is driven by an electric motor.

16. The hardness adjuster according to claim 8, wherein
the wire pulling device is a wire pulley which is driven by an electric motor.

17. The hardness adjuster according to claim 9, wherein
the wire pulling device is a wire pulley which is driven by an electric motor.

18. The hardness adjuster according to claim 10, wherein
the wire pulling device is a wire pulley which is driven by an electric motor.
